(19) United States
(12) Patent Application Publication
(10) Pub. No.: US 2006/0093575 A1
(43) Pub. Date: May 4, 2006

(54) Oxaliplatin anti-resistance agent

(75) Inventors: Bernard Pau, Teyran (FR); Isabelle Gourdier, Teyran (FR); Maguy Del Rio, Prades Le Lez (FR); Laure Crabbe, La Jolla, CA (US)

Correspondence Address: OBLON, SPIVAK, MCCLELLAND, MAIER & NEUSTADT, P.C., 1940 DUKE STREET, ALEXANDRIA, VA, 22314, US

(73) Assignees: CENTRE NATIONAL DE LA RECHERCHE SCIENTIFIQUE, Paris (FR); UNIVERSITE DE MONTPELLIER 1, Montpellier (FR); INSTITUT PASTEUR, Paris (FR)

(21) Appl. No.: 10/516,946

(22) PCT Filed: Jun. 17, 2003
(86) PCT No.: PCT/FR03/01842

§ 371 Date: Aug. 9, 2005

(30) Foreign Application Priority Data
Jun. 17, 2002 (FR) 02/07417

(51) Int. Cl. A61K 38/19; C12Q 1/68; G01N 33/574; A61K 31/33; A61K 31/553; A61K 31/444

(52) U.S. Cl. 424/85.1; 435/6; 435/7.23; 514/183; 514/211.08; 514/332

(57) ABSTRACT

The invention concerns a method for detecting in vitro resistance of cancer cells to an oxaliplatin treatment characterized in that it consists in measuring mitochondrial apoptosis of cancer cells being treated or capable of being treated or to be treated with oxaliplatin.

This invention relates to the treatment of cancer in patients presenting resistance to oxaliplatin.

The invention in particular relates to the diagnosis of resistance of colorectal cancers to the anti-tumoral medication “oxaliplatin” (international non-proprietary name of this product, the commercial name of which is Eloxatine).

The invention also relates to the reduction of this resistance by appropriate treatments using. “anti-resistance” agents, improving the effectiveness of the oxaliplatin-based treatment (in combination with oxaliplatin or by second intention, after development of oxaliplatin resistance).

Chemotherapeutic treatments of colorectal cancers, in spite of the availability of active anti-tumoral molecules like oxaliplatin, see their efficacy very limited by the frequent occurrence of resistance in tumor cells to the cytotoxic effects of the medications, used alone or in combination.

The reduction of this resistance is therefore a major issue for health care and the pharmaceutical industry. Anti-cancerous oxaliplatin treatments, the administration of which is aimed at destroying cancer cells, are in particular described in documents U.S. Pat. No. 5,716,968 and EP 0 943 331.

The attainment of this objective, amounting to the creation of “anti-resistance” treatments combined with anti-tumor medications like oxaliplatin, requires the identification of molecular mechanisms up to now not elucidated that govern the emergence of resistance inside tumor cells.

The identification of these mechanisms, unknown at present, of the resistance of cancers, in particular colorectal cancer, to oxaliplatin, therefore aims principally at two applications:

early diagnosis of resistance: it consists of avoiding chemotherapies that would have no therapeutic benefit, while they represent a toxic risk or high cost,

treatment by medications opposing or circumventing the resistance mechanisms.

It is important to note that in prior art there is no early test for resistance to oxaliplatin treatment.

Oxaliplatin resistance: oxaliplatin is a platin salt possessing an anti-tumor activity spectrum much broader than conventional platin salts such as cisplatin or carboplatin. The mechanisms of resistance to cisplatin have been for the most part elucidated, but do not take oxaliplatin resistance into account. More particularly, the deregulation of the MMR or NER repair systems associated with cisplatin resistance does not confer resistance to oxaliplatin. Oxaliplatin resistance remained unexplained until this invention. Oxaliplatin (CgH, 4N204Pt, [(1R, 2R)-1,2-cyclohexanediamine-N,N′] [oxalato (2-)-O,O′] platinum), is a diaminocyclohexane known to damage DNA. This invention covers resistance to oxaliplatin, as well as, should the occasion arise, to oxaplatin derivatives that also give rise to resistance.

Two studies, conducted on the same ovarian cancer cell model (line ATCC A2780), identified a potential effector mechanism for resistance to oxaliplatin in this type of cancer: an increase in intracellular glutathione, as well as a reduction of intracellular accumulation of platin and DNA-platin adducts, are associated with resistance to oxaliplatin. But these studies do not provide a functional demonstration of these identifications. The hypothesis implicating glutathione is emphasized in the document Cancer Lett. 1996 Jul. 19, 105(1):5-14, Altered Glutathione Metabolism in Oxaliplatin Resistant Ovarian Carcinoma Cells (Elakawi Z, Abu-hadid M, Perez R, Glavy J, Zdanowicz J, Creaven PJ, Pendyala L.), Department Of Investigational Therapeutics, Roswell Park Cancer Institute, Buffalo, N.Y. 14263, USA.

A hypothesis for the participation of DNA repair mechanisms is advanced in the document Cellular and Molecular Pharmacology of Oxaliplatin, Vol. 1, 227-235, January 2002, Molecular Cancer Therapeutic, (Eric Raymond, Sandrine Faivre, Stephen Chaney, Jan Woynarowski and Esteban Cvitkovic).

However, these studies do not make it possible to explain with certainty the mechanisms of resistance observed.

Thus, the invention aims at mitigating the disadvantages of prior art, and in particular at elucidating the mechanisms of resistance of the cancers, in particular colorectal cancers, to oxaliplatin, in order to be able to implement early diagnosis of resistance during treatment, and tailor a rational pharmacological approach that can lead to the development of “anti-resistance” treatments better targeted to these mechanisms.

Vice-versa, performance of early diagnostic tests for resistance will make it possible, at least, to inform the oncologist of the necessity of reorienting treatment (for example by introducing other medications in the therapeutic scheme). The benefit of this will be the reduction of adverse effects and the limitation of unnecessary health-care expenditures. Moreover, the availability of specific treatments (medications, gene therapies, etc.) opposing or circumventing resistance at the level of demonstrated mechanisms (mitochondrial apoptosis) will restore the efficacy of oxaliplatin. The benefit will obviously be medical but also economic: the gain in efficacy will justify the maintenance and extension of oxaliplatin use.

The inventors had to resolve several technical problems including the implementation of a reliable experimental model (selection and characterization of cell lines resistant to oxaliplatin from reference lines) and the exploration of this model (identification of the alteration of mitochondrial apoptosis as marker for specific resistance to oxaliplatin).

The inventors succeeded in showing that oxaliplatin resistance is associated with abnormal expression of the mitochondrial apoptosis genes. Prior art describes apoptosis inducing compounds acting directly and specifically at the mitochondrial level. However, the connection between mitochondrial apoptosis (MA) and mechanisms of oxaliplatin resistance is not at all described or suggested in prior art.

The inventors have therefore developed a method for the diagnosis of oxaliplatin resistance, based on the visualization of markers for the alteration of mitochondrial apoptosis in the tumor cells, by any appropriate means: biochemical such as immunodetection, genetic such as sequencing or the quantification of transcripts.

Thus, according to a first characteristic, the invention relates to a detection process, in vitro or in vivo, of the resistance of cancer cells to oxaliplatin treatment, comprising the measurement of mitochondrial apoptosis of cancer cells that are treated or can or should be treated with oxaliplatin. By resistance of cancer cells treated with oxaliplatin we mean that the cancer cells, of a patient or in culture, resist oxaliplatin treatment in such a way that this treatment is not totally satisfactory because it does not make it possible to destroy them to a sufficient extent.

This detection process relates in particular to colorectal cancers. However, other cancers whose treatment involves administration of oxaliplatin also belong to the invention, in particular certain cancers of the ovaries, the germinal cells, the lung, the digestive tract, the prostate, the pancreas, the small intestine and the stomach.

According to one realization the detection process involves the measurement of the expression of at least one gene of mitochondrial apoptosis. By “expression of at least one gene of mitochondrial apoptosis,” we mean the level of expression of at least one effector or marker gene of mitochondrial apoptosis. By effector gene, we mean a gene responsible at least in part for mitochondrial apoptosis, this expression being expressed in particular by the amount of RNA produced, the amount of protein coded by these genes, the level of activity of these proteins. For example, a low level of apoptosis can be due to the synthesis of an apoptosis protein whose sequence differs with respect to that of a non-resistant patient, the amount of protein being normal but its biological activity being lower. By marker gene we mean a gene that is not necessarily implicated in the mechanisms of mitochondrial apoptosis, but whose level of expression is correlated with a specified level of apoptosis.

Among the effector or marker genes for mitochondrial apoptosis, we can in particular analyze, in addition to the genes already studied by the inventors (Bax gene in particular), genes known for their implication in mechanisms of mitochondrial apoptosis, described in particular in document U.S. Pat. No. 6,268,398:

factors initiating or stimulating the apoptosis cascade and/or the activity of caspase proteases (Thornberry and Lazebnik, Science 281:1312-1316, 1998), such as cytochrome c, which are released following oxidative stress;

“apoptosis inducing factors” described in Murphy, Drug Dev. Res. 46:18-25, 1999;

factors inducing chromatin condensation (Marchetti et al., Cancer Res. 56:2033-38, 1996) which precede apoptosis;

Bcl-2 proteins, known for their anti-apoptosis activity, located in the outer mitochondrial membrane (Monaghan et al., J. Histochem. Cytochem. 40:1819-25, 1992), which protect the membranes against oxidative stress (Korsmeyer et al., Biochim. Biophys. Act. 1271:63, 1995; Nguyen et al., J. Biol. Chem. 269:16521-24, 1994) in particular by blocking the release of cytochrome c and the activation of caspase 3 (Yang et al., Science 275:1129-1132, 1997; Kluck et al., Science 275:1132-1136, 1997).

Those skilled in the art have at their disposal numerous appropriate techniques for the measurement of gene expression. We cite, for example:

the measurement of mRNA and cDNA, by means of RT-PCR techniques, Northern blot, hybridization to cDNA banks (Sambrook et al., Molecular Cloning—A Laboratory Manual, Cold Spring Harbor Press, New York (1989), techniques of differential display (Liang et al., 1995, Curr. Op. Immunol. 7:274-280; EP 534 858), techniques using cDNA probes or oligonucleotides (Eisen, M. B. and P. O. Brown, Methods Enzymol, 303:179-205 (1999); Brown, P. O. and D. Botstein, Nat Genet, 21 (1 Suppl):33-7 (1999); Cheung, V. G., et al., Nat Genet, 21(1 Suppl):15-9(1999));

measurement of proteins by means of western-blot and immunohistochemical analyses.

For example, the quantification of cytochrome c can make use of a spectrophotometric or immunochemical method. The release of cytochrome c from the mitochondria can be followed, for example, by means of immunological methods, by MALDI-TOF spectrometry coupled with affinity capture (in particular for apocytochrome c and holocytochrome c) and by the SELDI system (Ciphergen, Palo Alto, USA).

Measurement of the activity of caspases can make use of tests on caspase substrates (Ellerby et al., 1997 J Neurosci. 17:6165), such as the labeled synthetic peptide Z-Tyr-Val-Ala-Asp-AFC, Z being a benzoyl carbonyl group and AFC 7-amino-4-trifluoromethylcoumarin, on nuclear proteins such as UI-70 kDa and DNA-PKcs (Rosen and Casciola-Rosen, 1997, J. Cell. Biochem. 64:50; Cohen, 1997, Biochem. J. 326:1).

To the extent that an abnormally low level of mitochondrial apoptosis may be due to several genes, detection may involve the measurement of the level of expression of several genes for apoptosis; it is possible to determine in this way the profile of expression of several genes that are compared between patients for whom resistance has been diagnosed and non-resistant patients. By determining sufficiently precise profiles of expression, the clinician can detect a resistant phenotype, and also predict resistances in order to optimize the therapy.

The genes for mitochondrial apoptosis may belong to the mitochondrial DNA or to the nuclear DNA.

According to, one realization, the detection process involves the measurement of the amount of Bax protein in the cancer cells and the measurement of the mRNAs coding for the Bax protein.

According to one realization the detection process involves:

a) determination of the level of mitochondrial apoptosis and/or of the level of expression of at least one gene for mitochondrial apoptosis of cancer cells sampled from a patient treated with oxaliplatin;

b) comparison of the level of mitochondrial apoptosis with a control sample from a patient not resistant to oxaliplatin.

A lower level of mitochondrial apoptosis indicates resistance. A lower level of expression indicates resistance in the case of an effector gene stimulating mitochondrial apoptosis, a higher level of expression indicates resistance in the case of an effector gene inhibiting mitochondrial apoptosis.

Deviations in the levels of expression analyzed over a sufficient number of patients make it possible to determine the risk and the degree of resistance, the significant quantitative deviations observed being low or high according to the genes implicated.

It is possible to use samples for example from biopsies on an individual suffering from cancer at different times. For example, a first sample corresponds to the time of diagnosis and a second sample is obtained at a second time after treatment of the patient with a composition consisting of an anti-resistance agent. The diagnosis can also be carried out following gene therapy, for example to evaluate the level of mitochondrial apoptosis following the transfer of nucleic acid sequences coding for the proteins of mitochondrial apoptosis.

The invention also relates to a process for the detection of cancer cells resistant to oxaliplatin involving putting the biological sample examined together with at least one antibody capable of recognizing an apoptosis protein or a biologically active fragment of this protein, and the visualization of the antigen-antibody complex that may have formed.

For the implementation of this process a kit can be used consisting of:

a) an antibody that is for example monoclonal or polyclonal, said antibody being capable of recognizing an apoptosis protein or a biologically active fragment of this protein;

b) possibly reagents for the composition of a medium conducive to the immunological reaction;

c) possibly reagents making possible the visualization of antigen-antibody complexes produced by the immunological reaction.

In this way, antibodies can be used to detect under-expressed apoptosis proteins. Most preferably, for a given apoptosis protein, the antibodies recognize epitopes of the protein that are not present in other proteins.

Antibodies designed to specifically recognize one or more epitopes of apoptosis proteins, in particular the Bax protein, can in particular be monoclonal, polyclonal, humanized or chimeric antibodies, single chain antibodies, Fab fragments, Fab′2 fragments, fragments produced by an Fab expression bank and anti-idiotypic antibodies.

Monoclonal antibodies, a homogeneous population of antibodies for a specific antigen, can be obtained by means of techniques known to those skilled in the art, such as the hybridoma technique of Kohler and Milstein (Nature 256:495-497,1975; and U.S. Pat. No. 4,376,110), the technique of human B cell hybridomas (Kosbor et al., Immunology Today 4:72, 1983; Cole et al., Proc. Natl. Acad. Sci. USA 80:2026-2030, 1983), the technique of EBV hybridomas (Cole et al., “Monoclonal Antibodies and Cancer Therapy,” Alan R. Liss, Inc. pp. 77-96, 1985). It is also possible to prepare monoclonal antibodies by means of phage display bank kits marketed by Pharmacia or Stratagene.

Chimeric antibodies can be obtained according to a technique of Morrison et al., Proc. Natl. Acad. Sci., USA 81:6851-6855. Fab expression banks can be constructed according to the technique of Huse et al., Science 246:1275-1281, 1989. Anti-idiotypic antibodies can be obtained by the technique of Greenspan and Bona, FASEB J. 7:437-444, 1993.

According to another characteristic, the invention relates to a process for detection of the resistance of a cancer to oxaliplatin consisting of the in vitro or in vivo detection of at least one mutation indicative of defective apoptosis of cancer cells in the case of oxaliplatin treatment. The identification of such mutations makes possible early diagnosis that makes it possible to better target the therapy and to avoid inappropriate treatments. Comparative sequencing of apoptosis genes between patients with an early diagnosis of resistance and resistant patients can also be used. Thus the detection process can include for example the detection of a mutation in a region of the Bax gene containing a series of 8 deoxyguanines.

The invention also relates to a process for the detection of cancer cells resistant to oxaliplatin implementing at least one primer sequence or specific probe for a mitochondrial apoptosis gene such as the Bax gene, obtained by appropriate techniques of construction using sequences retrieved for example from GenBank.

The invention thus also relates to a process consisting of:

a)isolation of the mitochondrial DNA from the biological sample to be examined, or the procurement of a cDNA from the RNA of the biological sample or from the genomic DNA;

b) specific amplification of the DNA of a) by means of at least one primer for amplification of a mitochondrial apoptosis gene in particular of the Bax gene.

It is thus possible to use a kit for the diagnosis of oxaliplatin resistance consisting of means for extraction of the mitochondrial DNA of cancer cells, means for detection and amplification of mRNA of mitochondrial apoptosis genes, for example of the Bax gene, or of genomic DNA.

The invention also relates to a process consisting of:

a) putting together a nucleotide probe of a mitochondrial apoptosis gene such as the Bax gene and the biological sample analyzed, the nucleic acid of the sample having, as the case may be, been previously made accessible to hybridization, under conditions allowing hybridization of the probe and the nucleic acid of the sample,

b) visualization of the hybrid possibly formed.

It is possible to use a kit for diagnosis of oxaliplatin resistance consisting of:

a) at least one compartment suitable to contain and, as the case may be, containing a primer or a probe for a mitochondrial apoptosis gene such as the Bax gene;

b) possibly the reagents necessary for the implementation of a hybridization reaction;

c) possibly at least one primer and the reagents necessary for a DNA amplification reaction.

According to another characteristic the invention relates to a process that aims to determine if oxaliplatin treatment is to be pursued and/or completed, characterized in that it consists of:

a) obtaining at least two samples comprising cancer cells coming from the patient undergoing oxaliplatin treatment;

b) measurement of the level of mitochondrial apoptosis, for example by means of measurement of the expression of the Bax protein, in the samples;

c) continuation of treatment when the level of apoptosis does not decrease during treatment.

According to another characteristic, the invention relates to a process for selection of compounds that inhibit oxaliplatin resistance, designated as anti-resistance compounds, the process consisting of the measurement of the expression of at least one mitochondrial apoptosis gene before and after addition of a candidate compound to the oxaliplatin resistant cancer cells of a patient.

In vitro, the process can involve the addition of at least one candidate compound to oxaliplatin resistant cancer cells sampled from a patient, the comparison of the level of mitochondrial apoptosis and/or expression of apoptosis genes in the presence and absence of the compound, the deduction of the anti-resistance effect when the level of apoptosis is greater after addition of the compound. The anti-resistance effect is also deducted if the level of expression after addition of the compound is greater when the gene is a gene that stimulates apoptosis, and lesser when the gene is a gene that is inhibitory of mitochondrial apoptosis.

In vivo the selection process may include, in a patient treated with oxaliplatin and resistant to oxaliplatin:

a) obtaining at first of a first sample consisting of cancer cells of the patient;

b) administration of the candidate compound to the patient;

c) obtaining later of a second sample consisting of cancer cells of the same patient;

d) determination of the level of mitochondrial apoptosis and/or of the level of expression of at least a mitochondrial apoptosis gene such as the Bax gene in the first or second sample;

e) deduction of the oxaliplatin anti-resistance effect of the compound when the level of apoptosis is greater in the second sample.

The anti-resistance effect is also deducted if the level of expression is greater in the second sample when the gene is a gene that stimulates apoptosis, and lesser if the gene is a gene that inhibits mitochondrial apoptosis. Such in vivo procedures most preferably relate to compounds derived from compounds already identified as anti-resistant.

By anti-resistance agent we mean a compound capable of reducing, most preferably of totally offsetting, the oxaliplatin resistance of patients. These anti-resistance agents are designed to restore the normal level of expression of at least one mitochondrial apoptosis gene, either directly, or indirectly for example by activation or inhibition of molecules regulating the expression of these genes. An anti-resistance agent can for example block the activity of a compound responsible for abnormal inhibition of the activity of apoptosis genes at the level of transcription, translation, or activity of a protein.

Candidate compounds can be sought in particular among small molecules, polypeptides (for example oligopeptides, antibodies, antibody fragments) and nucleic acids. Targeting processes for oxaliplatin anti-resistance agents typically involve banks of molecules known to those skilled in the art such as banks of biological substances (in particular proteins) and banks of synthetic substances.

Banks of compounds can present themselves in solution form (e.g., Houghten, 1992, Biotechniques 13:412-421), on beads (Lam, 1991, Nature 354:82-84), on chips (Fodor, 1993, Nature 364:555-556). It is also possible to use banks described in the documents U.S. Pat. Nos. 5,292,646 and 5,270,281.

It is possible to study in particular the effect of compounds already known to those skilled in the art as stimulators of mitochondrial apoptosis, such as TNF (Tumor Necrosis Factor), FasL, glutamate, Herbimycin A (Mancini et al., J. Cell. Biol. 138:449-469, 1997), Paraquat (Costantini et al., Toxicology 99:1-2, 1995), protein kinase inhibitors such as Staurosporin, Calphostin C, derivatives of d-erythro-sphingosine, Chelerythrine chloride, inducers of MAP kinase such as Anisomycin and inducers of the MPT category to which the Bax protein belongs (Jurgenmeier et al., Proc. Natl. Acad. Sci. U.S.A. 95:4997-5002, 1998).

Among the tests that make it possible to measure the level of mitochondrial apoptosis, we may mention the measurement of the enzymatic activity of the mitochondrial complexes ETC I, II, III, IV and of ATP synthetase, the measurement of mitochondrial oxygen consumption (Miller et al., J. Neurochem., 67:1897, 1996), the measurement of the oxidation state of mitochondrial cytochrome c at 540 nm and the measurement of oxidative stress in the presence and absence of the anti-resistance agent.

According to another characteristic the invention relates to the use of at least one anti-resistance agent that stimulates mitochondrial apoptosis for the preparation of a medication in patients presenting or able to present resistance to oxaliplatin. By resistant patient we mean a patient presenting cancer cells resistant to oxaliplatin. Such an anti-resistance agent can be used in patients presenting a partial response to oxaliplatin treatment in order to improve the efficacy of the treatment.

According to one realization the anti-resistance compounds are derived from a selection process such as described previously. Those skilled in the art have at their disposal tests sufficiently well-described in the application to select these compounds; the invention therefore also covers the use of these compounds, even if the precise chemical structure of the compounds is not completely identified: if a tested compound fulfills the selection criteria (in particular stimulation of apoptosis, increase in expression of at least one apoptosis stimulating gene, reduction in expression of at least one apoptosis inhibiting gene), then those skilled in the art can use it for the preparation of a medication for anti-resistance to oxaliplatin without necessarily needing to know its chemical structure.

The treatment more specially targets cancer cells that have acquired oxaliplatin resistance. The treatment aims to restore a level of expression or activity of the genes implicated in mitochondrial apoptosis that is sufficient so that the resistant cancer cells more actively re-employ this process. Normal apoptosis is sought that is similar to that of non-resistant cancer cells, or at least an increase in mitochondrial apoptosis sufficient to reduce clinical symptoms.

Treatment of the patient will typically involve the combination of oxaliplatin and at least one anti-resistance agent, according to an administration that can be simultaneous, separate or spaced out in time. The amount of the anti-resistance agents to be administered to the patients must be sufficient to be therapeutically effective, in order to at least partially reduce oxaliplatin resistance. Treatment combining oxaliplatin and at least one agent of anti-resistance to oxaliplatin, in a resistant patient, aims preferentially at obtaining a therapeutic efficacy at least equal to that of oxaliplatin treatment in a non-resistant patient.

The invention also relates to a method for treatment of an oxaliplatin-resistant patient or one capable of presenting oxaliplatin resistance, involving the administration of at least one compound that stimulates mitochondrial apoptosis.

The invention also relates to a process for inhibiting oxaliplatin resistance in humans, involving the administration of a compound capable of selectively stimulating the mitochondrial apoptosis of cancer cells, in a patient requiring such an anti-resistance treatment.

The toxicity and therapeutic efficacy of anti-resistance agents can be determined by standard techniques of experimentation on cultured cells or laboratory animals. Transposition to human patients knowing these data is obtained by means of appropriate methods.

A formulation according to the invention consists of oxaliplatin typically in the amount of 1 to approximately 10 mg/ml, most preferably 1 to 5 mg/ml, and even more preferably from 2 to 5 mg/ml. The oxaliplatin doses administered to the resistant patient will typically be on the order of 10 mg/m2/day to 250 mg/m2/day, preferably 20 mg/m2/day to 200 mg/m2/day, most preferably between 50 and 150 mg/m2/day.

Administration may be repeated for cycles of 1 to 5 days spaced apart by an interval of 1 to 5 weeks. For patients presenting stronger resistance, the clinician will determine the appropriate oxaliplatin dose, the dose of anti-resistance agents and duration of treatment.

Should the occasion arise, the oxaliplatin and the anti-resistance agent can be combined with at least one compound known to those skilled in the art to reinforce the efficacy and/or stability of the oxaliplatin; such agents are described in the documents EP 0 943 331 and WO 01/66102.

The oxaliplatin will typically be combined with a pharmaceutically acceptable transporter, such as an appropriate solvent. The transporter will in general be water, or one or more solvents, or a mixture of water and one or more appropriate solvents. It might be preferred to use pure sterile water for injection, and among solvents: polyalkylene glycols such as polyethylene glycol, polypropylene glycol, polybutylene glycol and analogues, ethanol, 1-vinyl-2-pyrrolidone polymer, solutions of pharmaceutically acceptable sugars such as lactose, dextrose, sucrose, mannose, mannitol and cyclodextrins or analogues. The pH of the oxaliplatin solution formulations is typically 2 to 5, most preferably from 3 to 4.5.

The formulations of this invention are to be administered to patients by appropriate conventional routes, typically by the parenteral route (for example intravenous, intraperitoneal and analogues). Intravenous administration is performed for example over a period of 12 hours to 5 days. The percentage of the active compound in mixed formulations according to the invention comprising oxaliplatin and at least one resistance agent is adjusted according to the dosage and the degree of resistance to oxaliplatin in particular. The appropriate dosage for a particular patient is to be determined in particular as a function of the type of administration chosen, the duration of treatment, the size, the age, the physical condition of the patient, the degree of oxaliplatin resistance and the response of the patient to the composition.

Incorporation of the anti-resistance agent in the oxaliplatin composition is done by appropriate techniques.

For oral administration by means of pastilles, powders, granules and analogues, it is possible to use excipients such as lactose, sodium chloride, sucrose, glucose, urea, starch, calcium, kaolin, crystalline cellulose, salicylic acid, methyl cellulose, glycerol, sodium alginate, gum Arabic and analogues. It is possible to use usual binding agents such as glucose solutions, starch solutions and gelatin solutions. It is possible to use disintegrants such as starch, sodium alginate, agar powder and calcium carbonate. Among the absorbent agents, it is possible to use starch, lactose, kaolin and bentonite. Among the lubricants, it is possible to use purified talc, salts of stearic acids and polyethylene glycol.

Care will be taken in the formulation to avoid possible problems due to the combination of oxaliplatin and an anti-resistance agent such as problems with precipitation of the compounds.

A therapeutic oxaliplatin composition will typically contain 0.005% to 95%, most preferably 0.5 to 50% oxaliplatin-ahd anti-resistance agents.

On the plane of mechanism of action, according to one realization, the anti-resistance agent is an agent that stimulates the expression of at least one mitochondrial apoptosis gene.

According to one realization, the anti-resistance agent is a molecule capable of inhibiting expression of genes that inhibit mitochondrial apoptosis. It is possible to use complementary anti-sense oligonucleotides of mRNA coding for molecules inhibiting the expression of apoptosis effector genes. The anti-sense oligonucleotide will bind specifically to the mRNA of such inhibitory molecules, inhibiting their translation. The complementarity will have to be sufficient so that hybridization with the mRNA by the inhibitory molecule leads to the formation of stable hybrids. Typically, anti-sense strands will be used with a length between 6 and 50 nucleotides, typically of at least 10 to 20 nucleotides. The anti-sense strands can be synthesized by methods known to those skilled in the art, using nucleotides modified to increase the stability of the anti-sense/sense duplex. The following modified nucleotides can for example be used: 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, 5-methoxy-2-thiouracil, 3-(3-amino-3-N-2-carboxypropyl) uracil and 2,6-diaminopurine. The anti-sense strands can also be produced biologically by means of a vector for expression in which the anti-sense strand was sub-cloned in an anti-sense orientation.

Should the case arise, the anti-sense strand can be conjugated with peptide molecules facilitating its transport or activity at the level of the targeted site of action. It is possible to inject anti-sense molecules directly into a targeted area of the tissue and the anti-sense strand may be linked to molecules such as peptides or antibodies capable of binding specifically to receptors expressed on the surface of the target cells.

Administration of anti-sense strands is to be such that these molecules can act on an adequate level in the mitochondria.

According to another characteristic the invention relates to a pharmaceutical composition consisting of oxaliplatin and at least one anti-resistance agent capable of stimulating mitochondrial apoptosis, by stimulating expression of mitochondrial apoptosis genes or by blocking effectors responsible for resistance.

According to one realization, the anti-resistance agent is an agent for regulation-stimulation of expression of the Bax gene, and/or an agent for blocking of effectors of resistance.

Expression of mitochondrial apoptosis genes can be increased by transfer of nucleic acids containing a sequence coding for the apoptosis gene and/or a regulatory sequence, by means of transfer techniques appropriate for mitochondria. These sequences can be inserted in expression vectors and transferred into the cells, for example by means of plasmids. The nucleic acid inserted in the vector may code for the complete sequence of the apoptosis protein or a biologically active fragment with an activity most preferably of at least 50, 70, 90, 95% of the activity of the complete apoptosis protein.

The nucleic acids that can be used in the expression vectors can be operationally linked to regulatory sequences such as a promoter or enhancer sequence that stimulates their expression. These regulatory sequences can be those naturally associated with the genes coding for apoptosis proteins.

Those skilled in the art are aware of a large number of appropriate techniques for the transfer of nucleic acids into cells by vectors typically plasmids, such as the liposome-polybren technique, DEAE dextran transfection (Felgner et al., Proc. Natl. Acad. Sci. USA, 84:7413, 1987; Ono et al., Neurosci. Lett. 117:259, 1990; Brigham et al., Am. J. Med. Sci. 298:278, 1989), electropration (Neumann et al., EMBO J., 7:841, 1980), precipitation with calcium phosphate (Graham et al., Virology, 52:456, 1973; Wigler et al., Cell, 14:725, 1978; Feigner et al., supra), microinjection (Wolff et al., Science, 247:1465, 1990) and biolistic techniques. Most preferably, vectors appropriate for a transfer of genes at the mitochondrial level are to be used, for example HBV virus (hepatitis B Virus), the transfer described for example in the document U.S. Pat. No. 6,100,068.

Thus, treatment of oxaliplatin resistance rests on the use of new therapeutic processes, in particular resort to chemical substances and/or gene therapies, capable of reducing resistance by restoring activation of mitochondrial apoptosis normally caused by oxaliplatin in the tumor cells of colorectal cancers.

This invention also has for object a cell HCT116/S as registered on 16 Jun. 2003, under the number: I-3051, with the Collection Nationale de Cultures de Microorganismes (CNCM), Pasteur Institute, Paris, France.

The line designated as HCT116/S comes from a sub-cloning of the wild-type line HCT116 of the ATCC (in order to ensure the clonality of these cells). This line HCT116/S was registered on 16 Jun. 2003, under number: I-3051, with the Collection Nationale de Cultures de Microorganismes (CNCM) of the Pasteur Institute, Paris, France, under the terms of the Budapest Treaty.

This invention also has for purpose the use of cell HCT116/S such as registered with the CNCM on 16 Jun. 2003, under number: I-3051, or of any cell derived from this HCT116/S cell, to study the correlation between the resistance of cancer cells, most preferably colorectal, to anti-cancer treatment and the expression and/or activity of a mitochondrial apoptosis gene.

By cell derived from this HCT116/S cell as registered with the CNCM on 16 Jun. 2003, under number: I-3051, we mean to designate here in particular, any daughter cell originating from this line HCT116/S, or any variant cell originating from this line HCT116/S that has most preferably acquired resistance to a compound, such as an anti-cancer compound like oxaliplatin, or that has encountered a sensitivity to such a compound (cf. for example cell lines originating from HCT116/S described below in the paragraph “Implementation of the Experimental Model”).

Such HCT116/S cells as those registered with the CNCM on 16 Jun. 2003, under number: I-3051, or their derivative cells can also be used for the visualization and identification of a mitochondrial apoptosis gene whose expression is linked to the resistance of cancer cells, most preferably colorectal, to an anti-cancer treatment, in particular to treatment with oxaliplatin.

Such HCT116/S cells as those registered with the CNCM on 16 Jun. 2003, under number: I-3051, or their derivative cells can also be used for the selection of a compound capable of stimulating mitochondrial apoptosis of a cancer cell, said compound being designed to be combined with an anti-cancer agent to which said cancer cell is resistant, most preferably said anti-cancer agent to which said cancer cell is resistant being oxaliplatin and, as the case may be, said cell is a colorectal cancer cell.

Such a process involves in particular a step in which said compound to be tested and the anti-cancer agent to which said cancer cell is resistant are to be put together with said HCT116/S cells or their derivative cells, followed by observation of the resistance of these cells, in particular by studying the activity of mitochondrial apoptosis genes, such as the activity of Bax and/or Bak, or also of genes implicated in mitochondrial apoptosis such as cited above.

Other objects and advantages of the invention will appear upon reading of the detailed description that follows, illustrated by the following figures:

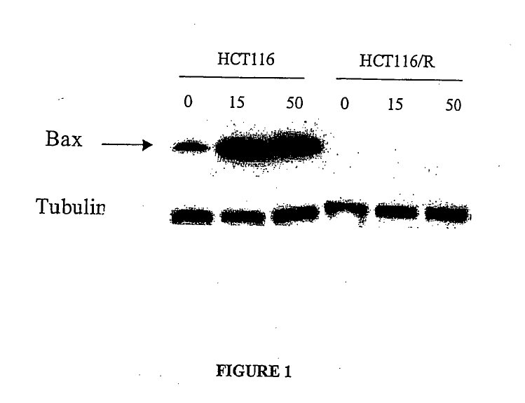

FIG. 1 shows that the HCT116R line, resistant to oxaliplatin, does not express the Bax protein;

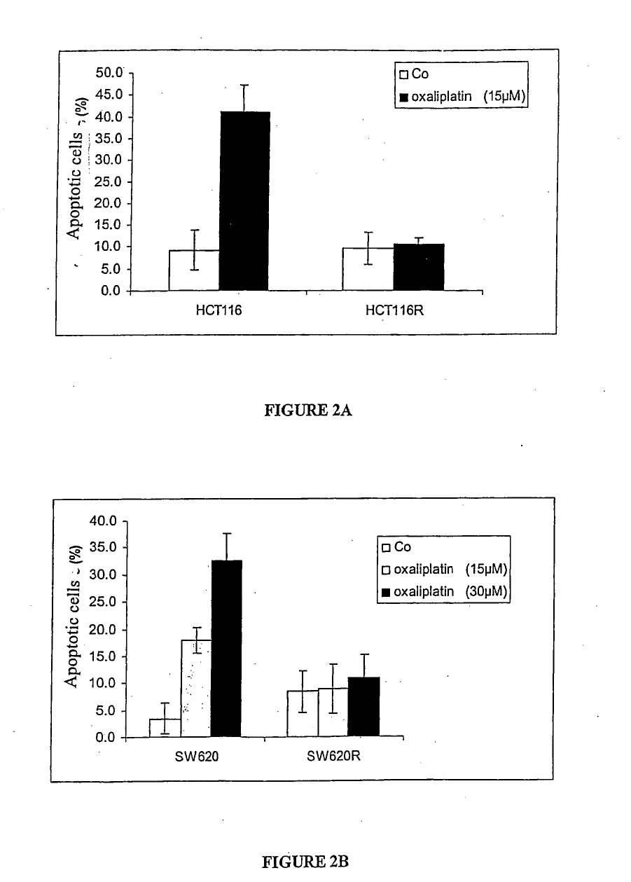

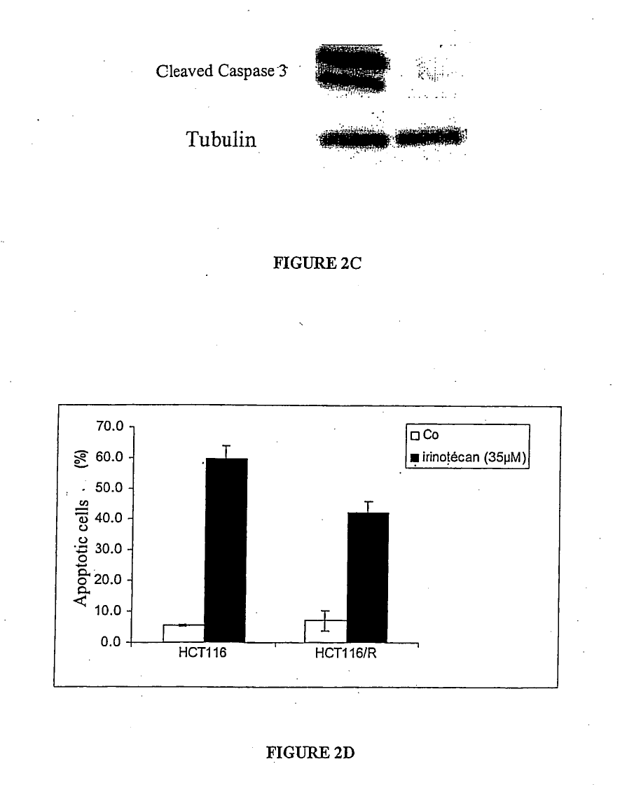

FIGS. 2A to 2D show that the HCT116R and SW620R lines resist apoptotic induction such as caused by oxaliplatin in the original HCT116 and SW620 lines;

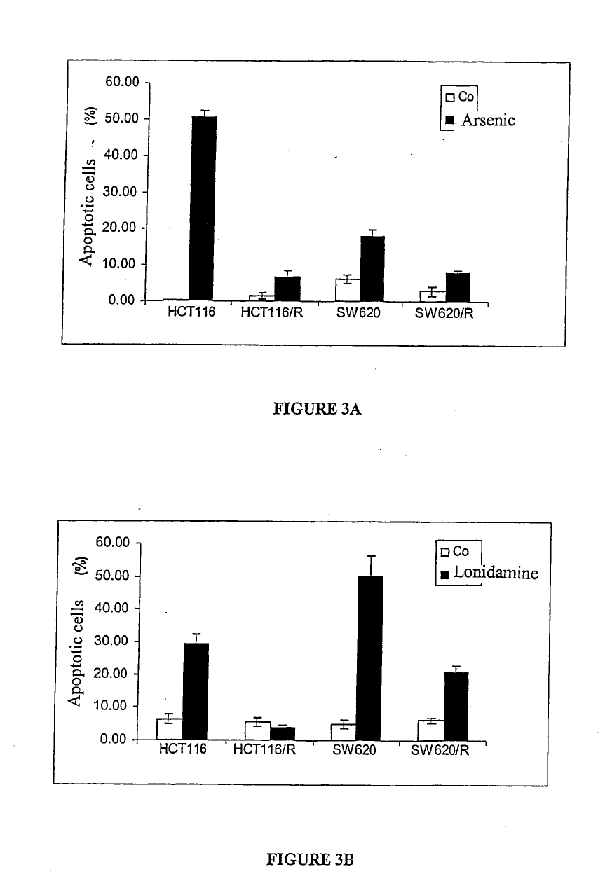

FIGS. 3A and 3B show that the HCT116R and SW620R lines also resist direct mitochondrial apoptotic induction, which can be obtained under the effect of the agents arsenic and lonidamine;

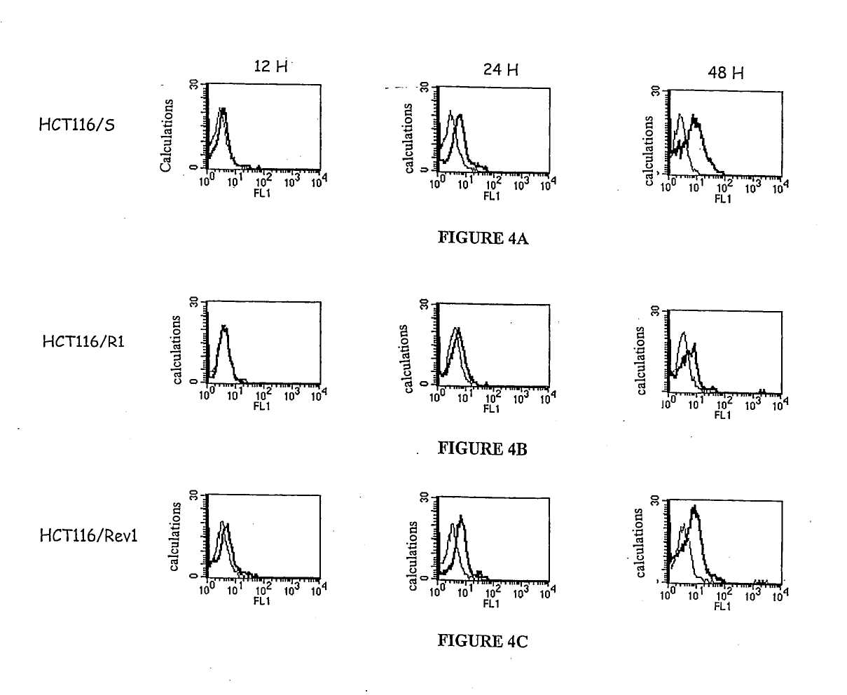

FIGS. 4A to 4C show that oxaliplatin sensitivity is associated with the degree of activation of Bax;

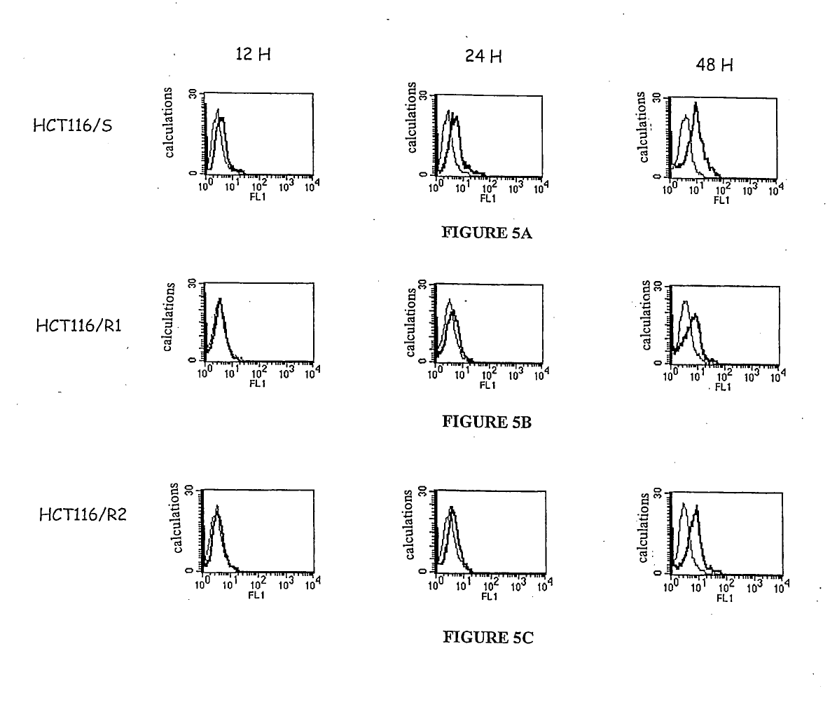

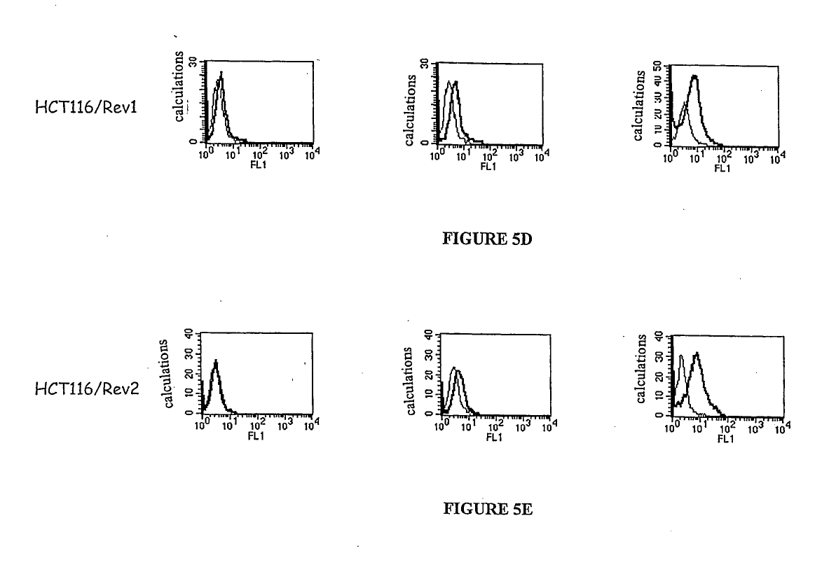

FIGS. 5A to 5E show that oxaliplatin sensitivity is associated with the degree of activation of Bak.

### IMPLEMENTATION OF THE EXPERIMENTAL MODEL

Work was performed “in vitro” on colorectal cancer cell lines (CRC) obtained from the international collection managed in the United States (ATCC) and re-cloned in the laboratory. These lines are referenced, in particular by the American Institute for Cancer Research (NCI/NIH), as standards for pharmacological evaluation of anti-tumor drugs.

From these lines, sensitive to the cytotoxic effect of oxaliplatin, the inventors have isolated derivative lines capable of specifically resisting oxaliplatin (and not the other medications cisplatin and irinotecan), by exposing these cells to increasing concentrations of oxaliplatin, in a scheme suitable for the acquisition of resistance. The results presented in the application relate to the original lines, HCT116 and SW620, as well as their derivatives HCT116R (also designated in the following as HCT116/R) and SW620R (also designated in the following as SW620/R), respectively 70 and 20 times more resistant than the original lines.

Concerning the referenced line ATCC HCT116, two lines are thus described in example 1 following:

the line designated HCT116, which is designated HCT116/S in example 2, originates from a sub-cloning of the wild-type line HCT116 of the ATCC (in order to ensure the clonality of these cells). This line HCT116/S was the object of registration on 16 Jun. 2003, under number: I-3051, in the Collection Nationale de Cultures de Microorganismes (CNCM) of the Pasteur Institute, Paris, France, according to the provisions of the Budapest Treaty, in accordance with Rule 6.1;

the line designated HCT116R or HCT116/R, which is designated HCT116/R2 in example 2, derived from the HCT116 line after acquisition of oxaliplatin resistance and cloning (corresponds to the clone 70 times more resistant than the sensitive reference line HCT116/S).

In example 2, three other cellular variants are described. They are:

the line HCT116/R1 derived from the line HCT116 after acquisition of oxaliplatin resistance and cloning (corresponding to a clone 30 times more resistant than the sensitive reference line HCT116/S),

the variant HCT116/Rev1 derived from the line HCT116/R1 after culture in the absence of oxaliplatin for 6 months. This variant is characterized by a return to the initial level of sensitivity (oxaliplatin sensitivity comparable to HCT116/S),

the variant HCT116/Rev2 derived from the line HCT116/R2 after culture in the absence of oxaliplatin for 15 months. This variant is characterized by an only partial loss of oxaliplatin resistance (variant HCT116/Rev2 is 16 times more resistant than line HCT116/S).

The lines HCT116/R1 and Rev1 do not carry the homozygous mutation of the Bax gene identified in HCT116/R2 (monitored by sequencing of the region containing codons 38 to 41). The level of expression of Bax is equivalent for HCT116/S, R1 and Rev1. The variant HCT116/Rev2 preserves the homozygous mutation identified in HCT116/R2 as well as the absence of Bax expression characteristic of this mutation.

### EXAMPLE 1

### Oxaliplatin Resistance in Lines HCT116R and SW620R Derived from CRC Lines HCT116 and SW620, and Results

TABLE 1
“Lines HCT116R and SW620R derived from CRC lines HCT116
and SW620 are specifically resistant to oxaliplatin”
IC₅₀(μM)^{a}

| Cell Line | Oxaliplatin | Cisplatin | | Irinotecan | |
|---|---|---|---|---|---|
| HCT116 | 0.32 ± 0.08(1.0)^{b} | 4.7 ± 1.8 | (1.0) | 7.7 ± 3.8 | (1.0) |
| HCT116/R | 21.9 ± 6.3 (68.4) | 13.4 ± 6.6 | (2.9) | 9.5 ± 4.2 | (1.2) |
| SW620 | 3.4 ± 0.6 (1.0) | 7 ± 1.7 | (1.0) | 23.3 ± 0.6 | (1.0) |
| SW620/R | 62.3 ± 12.9(18.3) | 9 ± 1.7 | (1.4) | 11.7 ± 2.5 | (0.5) |

^{a}The inhibitory concentration 50 or IC is the concentration of medication that reduces cell growth by 50%. The values of IC₅₀ were measured by wustl colorimetric test after incubation of the medication for 48 hours. The values correspond to the average ± SD obtained from at least three independent experiments.
^{b}The numbers between parentheses correspond to relative resistance, determined by the ratio of the IC₅₀ of the resistant clone divided by the IC₅₀ of the parental clone.

Table 1 shows that lines HCT116R and SW620R are approximately 70 times and 20 times more resistant to oxaliplatin than the lines from which they are derived. They present very little or no crossed resistance to cisplatin or irinotecan. Their resistance is therefore specific for oxaliplatin.

The study was conducted at the same time on two cell models of different genetic backgrounds so as to be able to reinforce the significance of the observed results; thus, line SW620 originates from a metastasis and possesses a mutated p53 regulatory protein whereas the HCT116 line originates from an early tumor with microsatellite instability and possesses a wild-type p53 protein. Observation of the alteration of mitochondrial apoptosis associated with the resistant phenotype (see below), in two different cellular contexts, makes it possible to generalize the results and therefore provide a high probability of its medical impact.

Exploration of the Experimental Model

The essential demonstrations were provided by these two distinct lines, so as to corroborate the universal character of the invention. Several complementary studies were limited to the HCT116 line and its derivative HCT116R.

The molecular mechanisms of oxaliplatin resistance of CRC cells being unknown, the inventors did a comparison study on the genic expression of the sensitive and resistant phenotypes in the HCT116 model (transcriptome analysis). The inventors succeeded in identifying a marked reduction in the levels of messenger RNA of certain genes linked to apoptosis, in particular of the Bax gene implicated in the path referred to as “Mitochondrial Apoptosis” (MA).

These observations were reinforced by biochemical analysis (immunoblotting indicates disappearance of Bax protein expression) and by sequencing of the Bax gene (in line HCT116R, a homozygous mutation of the Bax gene suppresses its expression).

FIG. 1:

FIG. 1 shows that the HCT116R line does not express the Bax protein, with or without oxaliplatin treatment, whereas the original HCT116 line expresses it without treatment and over-expresses it after oxaliplatin treatment. Sequencing showed that the HCT116R line is a homozygous mutant (deletion of a deoxyguanosine) in a region of the Bax gene containing a series of 8 deoxyguanosines (codons 38 to 41), which interdicts its expression by shifting of the reading frame. The original HCT116 line being heterozygous G8/G7, it therefore normally expresses the Bax gene.

Legend to FIG. 1: detection of Bax by Western blot in the absence of, or under the effect of treatment, with oxaliplatin in the HCT116 model. The cells are not treated or treated with oxaliplatin at a level of 15 μM for 48 hours (or 50 μM for 24 hours) before preparation of cell lysates. Tubulin expression is used as a control of equivalent protein deposits.

The inventors focused their work on the functional study of this path, in combination with oxaliplatin resistance. The principal results obtained are the following:

In the first place, the inventors showed that the lines HCT116R and SW620R, compared to the original lines, are resistant to induction of apoptosis by oxaliplatin. The inventors also verified in the HCT116 model that this resistance to apoptosis is specifically developed with respect to oxaliplatin, since the HCT116R line remains sensitive to induction of apoptosis by another anti-CRC medication (irinotecan) whose mechanism of action is different.

FIGS. 2A to 2D show that lines HCT116R and SW620R resist induction of apoptosis by oxaliplatin. This resistance was specifically developed with respect to oxaliplatin: the HCT116R line does not resist apoptotic induction caused an anti-CRC medication with a different mode of action, irinotecan (cf. FIGS. 2A, 2B, 2D). Resistance to induction of apoptosis by oxaliplatin, observed by cytofluorometry after labeling by annexine V, is confirmed by lack of activation of caspase 3 (FIG. 2C).

Legend to FIGS. 2A to 2D: cells HCT116 (and R) and SW620 (and R) are treated, for 48 hours prior to determination of the degree of apoptosis, by oxaliplatin (FIGS. 2A and 2B) or another anti-CRC medication, irinotecan, for cells HCT116 and HCT116R (FIG. 2D). A control is performed without contact with any medication (Co). The degree of apoptosis is then determined by cytofluorometry using labeling by annexine V. Independently, activation of the apoptosis effector protein Caspase 3 was evaluated in cells HCT116 and HCT116R after treatment for 24 hours with oxaliplatin in order to validate the entry into apoptosis of the cells as observed by cytofluorometry (FIG. 2C).

Subsequently, the inventors showed that the resistance to apoptosis induced by oxaliplatin in lines HCT116R and SW602R is accompanied by resistance to induction of apoptosis by two chemical agents known to be direct activators of MA (arsenic trioxide and lonidamine).

FIGS. 3A and 3B show that lines HCT116R and SW620R are resistant to apoptotic induction under the effect of the direct activators of MA arsenic and lonidamine.

Legend to FIGS. 3A and 3B: cells HCT116 (and R) and SW620 (and R) are treated, prior to determination of the degree of apoptosis, for 24 hours by arsenic trioxide (As), lonidamine (LND) or are left without treatment (control, Co). The degree of apoptosis is then determined by cytofluorometry after labeling with the dye “Mitocapture” which fluoresces differently in apoptotic cells and intact cells (with relation to mitochondrial integrity).

Thus the inventors showed that genetic, biochemical and functional alterations of apoptosis are associated with oxaliplatin resistance. They relate to two CRC cell lines separately selected for their specific resistance to oxaliplatin. The relevance of this selection is validated by the following characteristics:

The acquisition of oxaliplatin resistance is specific since it is not accompanied by acquisition of cisplatin resistance (a molecule that is related and that very frequently presents crossed resistance to oxaliplatin) or irinotecan resistance (another molecule indicated in the treatment of CRC as an alternative to oxaliplatin or in combination).

Oxaliplatin resistance, as well as functional alterations (resistance to apoptosis) is observed for an oxaliplatin concentration equivalent to the plasma peak in man during treatments.

The inventors have shown that resistance to apoptosis is exerted at the mitochondrial level. This is in particular shown by trials with direct inducers of MA. These alterations are therefore diagnostic markers for the resistance of colorectal cancers to oxaliplatin. Moreover, it is probable that pharmacological modulation of the MA pathway will make it possible to restore all or part of the sensitivity of CRCs to oxaliplatin. The inventors have verified, by a several months follow-up study of the derivative cell lines, that their resistance phenotype is spontaneously reversible in the absence of pharmacological pressure (=cell culture without oxaliplatin). This reversibility, total or partial according to the case, makes it possible to predict reversion under the effect of a substance opposing or circumventing the mechanisms of resistance within MA.

The inventors have moreover developed the purification of mitochondria from sensitive and resistant lines in order to isolate and test putative effectors of resistance (like PTPC), as well as agents blocking these effectors (anti-sense RNA, substances already known to block a physiological mechanism at the level of MA, etc.). The invention also covers the implementation of gene transfers restoring the phenotype of oxaliplatin sensitivity, and of processes for targeting of new chemical entities that make it possible to oppose or circumvent resistance, from effectors as targets and from banks of chemical substances as sources.

### EXAMPLE 2

### Activation of Bax and Bak

Activation of Bax

Bax is present in the cells in two forms: a latent (inactive) form and an active form that participates in the apoptotic process.

The overall level of Bax expression is equivalent for the lines HCT116/S, HCT116/R1 and HCT116/Rev1. Exposure to oxaliplatin induces an over-expression of Bax. This over-expression remaining comparable over all these lines, the inventors have tried to find out if the degree of Bax activation could account for the response of cells to oxaliplatin or if Bax definitely, taken by itself, couldn't be systematically associated with oxaliplatin sensitivity.

FIGS. 4A to 4C: Sensitivity to oxaliplatin associated with degree of Bax activation.

Legend to FIGS. 4A to 4C: Detection of activation of Bax by an antibody specific for the active conformation of Bax and intracellular analysis by flow cytometry. The cells are fixed, permeabilized and then incubated with the antibody specific for the active conformation of Bax. Finally visualization is carried out by incubation with the secondary antibody coupled with FITC. The labeling of untreated cells is shown by the curve in fine black line. That of treated cells (12, 24 or 48 hours with 15 μM oxaliplatin) is shown by the curves in thick black line. The appearance of strongly labeled cells (curves in thick black line shifted to the right) gives evidence of activation of Bax. The experiment was reproduced three times and comparable results were obtained.

The inventors have demonstrated by means of FIGS. 4A to 4C that the state of resistance or sensitivity of cells to oxaliplatin correlates well with the degree of activation of Bax. The activation is reduced and delayed in the resistant line HCT116/R1 (FIG. 4B) compared to the sensitive line HCT116/S (FIG. 4A). Return to the state of sensitivity of the revertant HCT116/Rev1 (FIG. 4C) is accompanied by return to early activation of Bax.

Activation of Bak

The pro-apoptotic molecule Bak plays a preponderant role in the same way as Bax in apoptosis mediated mitochondrial permeabilization. These two molecules seem to have functions that are very closely related and sometimes redundant. The inventors have therefore also tried to find out if there is a relationship between the level of activation of Bak and a response to oxaliplatin over all lines including the lines HCT116/R2 and Rev2 mutated in Bax.

FIGS. 5A to 5E: Oxaliplatin sensitivity associated with the degree of activation of Bak.

Legend to FIGS. 5A to 5E: Detection of activation of Bak by an antibody specific for the active conformation of Bak and intracellular analysis by flow cytometry. The cells are fixed, made permeable and then incubated with the antibody specific for the active conformation of Bax. Finally visualization is carried out by incubation with the secondary antibody coupled to FITC. Labeling of untreated cells is shown by the curve in the fine black line. That of treated cells (12, 24 or 48 hours with 15 μM oxaliplatin) is shown by the curves in the thick black line. The appearance of strongly labeled cells (curves in the thick black line shifted to the right) gives evidence of activation of Bak. This experiment was reproduced two times.

The inventors have demonstrated by means of FIGS. 5A to 5E that the activation of Bak in response to oxaliplatin treatment is delayed in the resistant lines HCT116/R1 (FIG. 5B) and HCT116/R2 (FIG. 5C). The revertants Rev1 (FIG. 5D) and Rev2 (FIG. 5E) repeat the level of activation of Bak of the sensitive line HCT116/S (FIG. 5A). As is the case with Bax, activation of Bak correlates well with the response of the cells to oxaliplatin.

In conclusion, a co-activation of Bax and Bak can be observed in response to oxaliplatin. Early activation of Bax and Bak is associated with the state of sensitivity of the cells to oxaliplatin.

1. Process for in vitro detection of resistance of cancer cells to oxaliplatin treatment, characterized in that it involves the measurement of the mitochondrial apoptosis of cancer cells that are treated or can or are to be treated with oxaliplatin.

2. Process according to claim 1, characterized in that the cancer is a cancer treated with oxaliplatin, in particular a colorectal cancer, a cancer of the ovaries, a cancer of the germinal cells, a cancer of the lung, a cancer of the digestive tract, a cancer of the prostate, a cancer of the pancreas, a cancer of the small intestine or a cancer of the stomach.

3. Process according to claim 1 or 2, characterized in that it involves the measurement of the expression of at least one mitochondrial apoptosis gene.

4. Process according to any of claims 1 to 3, characterized in that it involves the measurement of the expression of at least one gene coding for a Bax, Bcl-2 or cytochrome c protein.

5. Process according to claim 3 or 4, characterized in that it involves the measurement of mRNA transcripts of the mitochondrial apoptosis genes.

6. Process according to claim 3 or 4, characterized in that it involves measurement of the amount and/or activity of mitochondrial apoptosis proteins in the cancer cells.

7. Process for in vitro detection of the resistance of cancer cells to oxaliplatin treatment characterized in that it involves the detection of at least one mutation indicative of deficient mitochondrial apoptosis in the case of treatment with oxaliplatin, in particular of a mutation in a region of the Bax gene containing a series of 8 deoxyguanines.

8. Process according to any of claims 1 to 6, characterized in that it involves:
a) determination of the level of mitochondrial apoptosis, and/or the level of expression of at least one mitochondrial apoptosis gene, in cancer cells sampled from a patient;
b) comparison to the level measured with a control sample of cells not resistant to oxaliplatin.

9. Process according to claim 6, characterized in that it involves putting cancer cells together with an antibody capable of recognizing a mitochondrial apoptosis protein or a biologically active fragment, and the visualization of the antigen-antibody complex possibly formed.

10. Process according to any of claims 1 to 5, characterized in that it implements a primer or probe sequence specific for the mitochondrial apoptosis gene.

11. Process according to claim 10, characterized in that it involves:
a) possible isolation of mitochondrial DNA from the biological sample to be examined, or the obtaining of a cDNA from the RNA of the biological sample or from genomic DNA;
b) specific amplification of the DNA from a) by means of at least one primer for amplification of the mitochondrial apoptosis gene.

12. Process according to claim 10, characterized in that it involves:
a) putting a nucleotide probe of an apoptosis gene together with the biological sample analyzed, the nucleic acid of the sample having, if need be, been previously made accessible to hybridization, under conditions allowing hybridization of the probe and the nucleic acid of the sample;
b) visualization of the hybrid possibly formed.

13. Process for selection of compounds that inhibit the resistance of cancer cells to oxaliplatin, characterized in that it involves:
a) addition of at least one candidate compound to the cancer cells resistant to oxaliplatin;
b) comparison of the level of mitochondrial apoptosis and/or expression of at least one apoptosis gene in the presence and absence of the compound;
c) deduction of the anti-resistance effect when the level of mitochondrial apoptosis is greater after addition of the compound, or when the level of expression is greater when the gene is a gene that stimulates mitochondrial apoptosis, or when the level of expression is less when the gene is a gene that inhibits mitochondrial apoptosis.

14. Use of at least one agent stimulating mitochondrial apoptosis, in particular chosen from among TNF, FasL, glutamate, Herbimycin A, Paraquat, inhibitors of protein kinase such as Staurosporine, Calphostin C, derivatives of d-erythro-sphingosine, Chelerythrine chloride, inducers of MAP kinase such as Anisomycin and inducers of MPT for the preparation of a medication for patients presenting or capable of presenting oxaliplatin resistance.

15. Use according to claim 14 for the preparation of a medication against colorectal cancer, or cancer of the ovaries, the germinal cells, the lung, the digestive tract, the prostate, the pancreas, the small intestine or the stomach.

16. Use according to claim 14 for the preparation of a medication against colorectal cancers.

17. Product containing oxaliplatin and an agent stimulating mitochondrial apoptosis, in particular chosen from among TNF, FasL, glutamate, Herbimycin A, Paraquat, inhibitors of protein kinase such as Staurosporine, Calphostin C, derivatives of d-erythro-sphingosine, Chelerythrine chloride, inducers of MAP kinase such as Anisomycin and inducers of MPT as a combination product for simultaneous use, separated or spaced apart in time as an anti-cancer agent.

18. Composition consisting of oxaliplatin and at least one anti-resistance agent capable of stimulating mitochondrial apoptosis, chosen from among TNF, FasL, glutamate, Herbimycin A, Paraquat, inhibitors of protein kinase such as Staurosporine, Calphostin C, derivatives of d-erythro-sphingosine, Chelerythrine chloride, inducers of MAP kinase such as Anisomycin and inducers of MPT.

19. Kit for diagnosis of resistance of a cancer to oxaliplatin characterized in that it includes:
a) at least one compartment suitable to contain a probe;
b) possibly the reagents necessary for the implementation of a hybridization reaction;
c) possibly at least one primer and the reagents necessary for a DNA amplification reaction.

20. Cell HCT116/S as registered on 16 Jun. 2003, under number: I-3051, with the Collection Nationale de Cultures de Microorganismes (CNCM), Pasteur Institute, Paris, France.

21. Use of cell HCT116/S according to claim 20, or of any cell derived from this cell HCT116/S, to study the correlation between the resistance of cancer cells, most preferably colorectal, to anti-cancer treatment and the expression of a mitochondrial apoptosis gene.

22. Use of cell HCT116/S according to claim 20, or of any cell derived from this cell HCT116/S, for the visualization and identification of a mitochondrial apoptosis gene whose expression is linked to the resistance of cancer cells, most preferably colorectal, to anti-cancer treatment.

23. Use of cell HCT116/S according to claim 20, or of any cell derived from this cell HCT116/S, for the selection of a compound capable of stimulating mitochondrial apoptosis in a cancer cell, said compound being designed to be combined with an anti-cancer agent to which said cancer cell is resistant, most preferably said anti-cancer agent to which said cancer cell is resistant being oxaliplatin and, as the case may be, said cell is a colorectal cancer cell.
